(19) United States
(12) Patent Application Publication
(10) Pub. No.: US 2021/0315842 A1
(43) Pub. Date: Oct. 14, 2021

(54) STABLE PHARMACEUTICAL COMPOSITIONS FOR PRESSURIZED METERED DOSE INHALERS

(71) Applicant: LUPIN INC., Coral Springs, FL (US)

(72) Inventors: Mukul DALVI, Coral Springs, FL (US); Abhishek GUPTA, Coral Springs, FL (US); Agnes COLOMBANI, Coral Springs, FL (US)

(21) Appl. No.: 16/972,399

(22) PCT Filed: Jun. 4, 2019
(86) PCT No.: PCT/US2019/035362

§ 371 Date: Dec. 4, 2020

Related U.S. Application Data
(60) Provisional application No. 62/680,173, filed on Jun. 4, 2018.

(51) Int. Cl. A61K 31/137; A61K 47/06; A61K 47/10; A61M 15/00; A61K 31/575; A61K 47/12; A61K 31/4748; A61K 31/401

(52) U.S. Cl. CPC A61K 31/137; A61K 47/06; A61K 47/10; A61K 31/401; A61K 31/575; A61K 47/12; A61K 31/4748; A61M 15/0001

(57) ABSTRACT

The present invention provides a stable pharmaceutical composition to be used with pressurized metered dose inhalers and comprises a β2 agonist, a propellant, a co-solvent, an organic acid(s) and optionally water. The invention further provides stable pharmaceutical composition comprising β2 agonist, an inhaled corticosteroid and/or a long acting muscarinic antagonist. The invention also provides pharmaceutical composition for the treatment or prophylaxis of asthma, chronic obstructive pulmonary disease (COPD), rhinitis or as adjunct therapy for cystic fibrosis, non-cystic fibrosis bronchiectasis, lung infections or pulmonary fibrosis.

### CROSS REFERENCE TO RELATED APPLICATIONS

This application is a National Stage Application of PCT/US2019/035362, filed Jun. 4, 2019, which claims priority to U.S. Provisional Patent Application No. 62/680,173, filed Jun. 4, 2018, the disclosures of which are hereby incorporated by reference herein in their entireties. To the extent appropriate, a claim of priority is made to each of the above disclosed applications.

### FIELD OF THE INVENTION

The present invention provides a stable pharmaceutical composition comprising a β2 agonist, or a combination of a β2 agonist and an inhaled corticosteroid and/or a long acting muscarinic antagonist, a propellant, a co-solvent, an organic acid(s) and optionally water.

### BACKGROUND OF THE INVENTION

Metered dose inhalers (MDIs) are important and well known devices used to deliver appropriate therapy for a growing number of respiratory diseases. MDIs are a convenient, inexpensive delivery system and are widely used. Today, more than 20 different metered dose inhalers based products are on the market to deliver active pharmaceutical ingredients (APIs) for local and/or systemic therapy.

MDIs when actuated create propellant droplets containing the pharmaceutical product for delivery to the respiratory tract as an aerosol. Formulations for aerosol administration via MDIs can be solutions or suspensions. Solution formulations offer the advantage of being homogeneous, with the active ingredient and excipients completely dissolved in the propellant vehicle or its mixture with suitable co-solvents such as ethanol. Solution formulations also obviate physical stability problems of micronized particles associated with suspension formulations so assuring more consistent and uniform dosage administration. Since solution formulations provide smaller mass median aerodynamic diameters (MMADs) they facilitate delivery to the entire lung including the alveolar regions. The mass median aerodynamic diameter is generally measured with a cascade impactor.

However, a significant challenge associated with solution formulations is the chemical stability of the active ingredients. The loss of active substance content should be minimized during the shelf-life and the appearance of degradative product should be diminished so that the medicament can be used with its effectiveness and safety. It means, the physical and chemical stability as well as the maintenance of the quality parameters during the shelf-life of the aerosol are of essential importance for its practical medical use.

There has been a common challenge in preparing solution formulations containing β2-agonist because they are often chemically unstable especially in the presence of co-solvents such as ethanol which are necessary to formulate the solutions. Thus, the investigations have been directed vastly to avoid the degradation of β2-agonist.

EP 1787639 B1 discloses an aerosol solution composition which comprises a β2-agonist drug of the phenylalkylamino class bearing a functional group sensitive to oxidative and/or hydrolytic reaction in a solution of a liquefied HFA propellant, a co-solvent selected from pharmaceutically acceptable alcohols, wherein the apparent pH of the solution is comprised between 2.5 and 5.0 by addition of small amounts of a mineral acid selected from hydrochloric, nitric and phosphoric acid, wherein the active ingredient is formoterol as β2-agonist drug or a salt thereof in combination with a steroid selected from beclometasone dipropionate, fluticasone propionate, budesonide and its 22R-epimer or an anticholinergic atropine like derivative selected from ipratropium bromide, oxitropium bromide and tiotropium bromide.

EP 2223682 B1 discloses an aerosol composition which consists of an active ingredient formoterol fumarate in combination with beclometasone diproprionate in a solution of a liquefied HFA 134a propellant and 12% w/w ethanol as a co-solvent, and hydrochloric acid in an amount such that the solution has an apparent pH between 3.0 and 3.5.

EP 2 010 190 discloses a pressurized solution formulation for a metered dose inhaler comprising formoterol fumarate in combination with beclometasone dipropionate as active substances dissolved in a mixture consisting of HFA134a propellant and an amount of ethanol of 12% w/w as a co-solvent and 0.024% w/w hydrochloric acid (1M) for use in the prevention and/or treatment of a severe broncho-pulmonary disease selected from severe persistent asthma or severe or very severe chronic obstructive pulmonary disease (COPD), wherein, upon actuation of said inhaler, 50 μl of said solution comprising formoterol fumarate at a dose per actuation of 6 μg and beclometasone dipropionate at a dose of 100 μg per actuation are metered for delivery.

EP 1 660 035 discloses a pharmaceutical compositions are propellant free solutions intended for nebulization.

US 2006/0140873 discloses a composition for use in a metered dose inhaler (MDI), the composition comprising predetermined amounts of an active pharmaceutical ingredient (API) insoluble in the composition, a propellant comprising a hydrofluoroalkane (HFA), and a pharmaceutically acceptable non-aminated C1-6 organic acid that increases post-shaking suspension time of the API in the composition to at least 30 seconds to provide uniform dosing of the API from the inhaler over at least 30 seconds post-shaking.

The present invention discloses a pharmaceutical composition comprising to be used with pressurized metered dose inhalers and comprises of one or a combination of active ingredients, specifically a β2 agonist or a combination of a β2 agonist and an inhaled corticosteroid and/or a long acting muscarinic antagonist, a propellant, a co-solvent, an organic acid(s) and optionally water.

### SUMMARY OF THE INVENTION

According to one aspect of the present invention there is provided a pharmaceutical composition comprising β2-agonist, propellant, a co-solvent and an organic acid(s).

According to another aspect of the present invention there is provided a pharmaceutical composition comprising β2-agonist, propellant, a co-solvent, an organic acid(s) and water.

According to one aspect of the present invention there is provided a pharmaceutical composition comprising long acting β2-agonist, propellant, a co-solvent and an organic acid(s).

According to one aspect of the present invention there is provided a pharmaceutical composition comprising short acting β2-agonist, propellant, a co-solvent and an organic acid(s).

According to one aspect of the present invention there is provided a pharmaceutical composition comprising long acting β2-agonist, propellant, a co-solvent, an organic acid(s) and water.

According to one aspect of the present invention there is provided a pharmaceutical composition comprising short acting β2-agonist, propellant, a co-solvent, an organic acid(s) and water.

According to one aspect of the present invention there is provided a pharmaceutical composition comprising formoterol fumarate, HFA134a, maleic acid, and ethanol.

According to one aspect of the present invention there is provided a pharmaceutical composition comprising formoterol fumarate, HFA227ea, maleic acid, and ethanol.

According to one aspect of the present invention there is provided a pharmaceutical composition comprising formoterol fumarate, HFA134a, maleic acid, ethanol and water.

According to one aspect of the present invention there is provided a pharmaceutical composition comprising formoterol fumarate, HFA227ea, maleic acid, ethanol and water.

According to one aspect of the present invention there is provided a pharmaceutical composition comprising β2-agonist, corticosteroid, propellant, a co-solvent and an organic acid(s).

According to one aspect of the present invention there is provided a pharmaceutical composition comprising β2-agonist, corticosteroid, propellant, a co-solvent, an organic acid(s) and water.

According to one aspect of the present invention there is provided a pharmaceutical composition comprising formoterol fumarate, beclometasone dipropionate, HFA134a, ethanol and maleic acid.

According to one aspect of the present invention there is provided a pharmaceutical composition comprising formoterol fumarate, beclometasone dipropionate, HFA227ea, ethanol and maleic acid.

According to one aspect of the present invention there is provided a pharmaceutical composition comprising formoterol fumarate, beclometasone dipropionate, HFA134a, ethanol, maleic acid and water.

According to one aspect of the present invention there is provided a pharmaceutical composition comprising formoterol fumarate, beclometasone dipropionate, HFA227ea, ethanol, maleic acid and water.

According to one aspect of the present invention there is provided a pharmaceutical composition comprising β2-agonist, anticholinergic drug, propellant, a co-solvent and an organic acid(s).

According to one aspect of the present invention there is provided a pharmaceutical composition comprising β2-agonist, anticholinergic drug, propellant, a co-solvent, an organic acid(s) and water.

According to one aspect of the present invention there is provided a pharmaceutical composition comprising long acting β2-agonist, corticosteroid, anticholinergic drug, propellant, a co-solvent and an organic acid(s).

According to one aspect of the present invention there is provided a pharmaceutical composition comprising long acting β2-agonist, corticosteroid, anticholinergic drug, propellant, a co-solvent, an organic acid(s) and water.

According to one aspect of the present invention there is provided a pharmaceutical composition comprising formoterol fumarate, beclometasone dipropionate, Glycopyrrolate, HFA134a, ethanol, maleic acid and water.

According to one aspect of the present invention there is provided a pharmaceutical composition comprising formoterol fumarate, beclometasone dipropionate, Glycopyrrolate, HFA227ea, ethanol, maleic acid and water.

### DETAILED DESCRIPTION

The present invention relates to a pharmaceutical composition comprising β2-agonist, a propellant, a co-solvent, an organic acid(s) and optionally water. The pharmaceutical composition is a solution or a suspension, preferably a solution.

The aerosol medicinal products are very important as pharmaceutical dosage forms for drug administration by pulmonary route. The physical and chemical stability as well as the maintenance of the quality parameters, during the shelf-life of the aerosol, are of essential importance for its practical medical use.

Solution compositions eliminate the typical concerns around physical stability of micronized particles required for lung delivery and ensure that a consistent particle size is achieved through the shelf-life of the product. Solution compositions also provide smaller Mass Median Aerodynamic Diameter (MMAD) and therefore facilitate delivery to the entire lung including the alveolar regions.

There has been a common challenge in preparing solution compositions containing β2-agonist as one of the actives because they are often chemically unstable especially in the presence of co-solvents such as ethanol which are necessary to formulate the solutions. Thus, the investigations have been directed vastly to avoid the degradation of β2-agonist.

The preparation of stable solution formulations is even more vital when bronchodilator β2-agonists having benzylic hydroxyl group, like formoterol, albuterol, and others, may suffer from inherent chemical stability due to their susceptibility to substitution by nucleophilic species in the formulation via SN1 or SN2 reactions. Ethanol can satisfy the role of a nucleophile and cause degradation of β2-agonists having benzylic hydroxyl group.

Surprisingly, it has been found that the organic acids such as maleic acid, which have much weaker proton donating ability, can stabilize β2-agonist in solution formulations containing co-solvent such as ethanol for delivery using pressurized metered dose inhalers.

The present invention provides a pharmaceutical composition comprising β2-agonist, a propellant, a co-solvent, an organic acid(s) and optionally water.

When water is present in the pharmaceutical compositions, it may present up to 5% by wt, more preferably up to 3% by wt, and the most preferably up to 1% by wt.

In accordance with the invention, β2-agonist is present in the composition in an amount between approximately 0.005% by weight and 1% by weight.

In accordance with the invention, the propellant of the invention include, but not limited to, HFA 134a (1,1,1,2-tetrafluoroethane), HFA 227ea (1,1,1,2,3,3,3-heptafluoropropane), HFA 152a (1,1-difluoroethane) or mixtures thereof.

The co-solvent of the invention include, but not limited to dichloromethane, chloroform, ethylacetate, N-methyl pyrrolidone, benzylalcohol, isopropylacetate, acetonitrile, tetrahydrofuran, isopropanol, methanol, ethanol or mixtures thereof. Preferably the co-solvent is ethanol; and it is present in an amount between approximately 1% by weight and 40% by weight, more preferably between 4% by weight and 20% by weight.

In accordance with the invention, “the organic acid” term refers to any organic compound with acidic properties. An organic acid is different from an inorganic acid (or mineral acid) that is derived from one or more inorganic compounds. Organic acids tend to have weaker proton donating ability than inorganic acids. The organic acid of the invention include, but are not limited to, maleic acid, fumaric acid, citric acid, acetic acid, xinafoic acid, oxalic acid, lactic acid, 2-methyl propionic acid, malic acid, butanoic acid, tartaric acid, propionic acid, pentanoic acid, succinic acid, glycolic acid, hexanoic acid, malonic acid, glutaric acid, formic acid, adipic acid, ascorbic acid, benzoic acid, glucuronic acid or mixtures thereof.

Preferably the organic acid is maleic acid; and it is present in an amount between approximately 0.001 and 1% by weight, more preferably between 0.001% by weight and 0.10% by weight.

The pharmaceutical composition further comprises a second active agent. The second active agent is a corticosteroid or long acting muscarinic receptor antagonist (LAMA).

The corticosteroid of the invention include, but not limited to, beclomethasone or beclometasone dipropionate, budesonide, ciclesonide, flunisolide, fluticasone propionate, fluticasone furoate, mometasone furoate, triamcinolone acetate, or prednisolone. Preferably the corticosteroid is beclometasone or beclometasone dipropionate; and it is present in an amount between approximately 0.001% by weight and 1% by weight, more preferably between 0.05% by weight and 0.30% by weight.

The long acting muscarinic receptor antagonist (LAMA) of the invention include, but not limited to, umeclidinium, aclidinium, glycopyrronium, ipratropium, oxitropium, tiotropium or pharmaceutically acceptable salts thereof. Preferably the long acting muscarinic receptor antagonist (LAMA) is glycopyrronium, and it is present in an amount between approximately 0.001% by weight and 1% by weight, more preferably between 0.05% by weight and 0.30% by weight.

Another embodiment provides the pharmaceutical composition comprising β2-agonist, a propellant, a co-solvent, an organic acid(s) and optionally water filled in a container having part or all of its internal metallic surfaces made of stainless steel, anodised aluminium or lined with an inert organic coating.

Another embodiment provides the use of pharmaceutical composition comprising β2-agonist, a propellant, a co-solvent, an organic acid(s) and optionally water for the treatment or prophylaxis of asthma, COPD, rhinitis or as adjunct therapy for cystic fibrosis, non-cystic fibrosis bronchiectasis, lung infections or pulmonary fibrosis.

In accordance with the present invention, composition of the present invention are less prone to hydrolytic and/or oxidative degradation. Thus, the most physically and chemically stable formulations of the invention have a significant decrease in total degradation of product. In addition to the economic advantages, the formulations in accordance with the present invention remain stable at the range of temperatures to which these medicaments are normally exposed. The person having ordinary skill in the art can also easily determine the presence of hydrolytic and/or oxidative degradation products, for example, by HPLC analysis.

The aerosol container consists of a metal canister and a measuring dosage valve having diverse plastic (typically polyester or polyamide), metal and elastomer surfaces. The use of coated containers has been found to confer an additional protection not only for the chemical stabilization of active substance but also for the absence of corrosion or other unacceptable deterioration signs of the container material by contact with the product.

The following examples are included to demonstrate particular embodiments of the invention. It should be appreciated by those of skill in the art that the techniques disclosed in the examples which follow represent techniques discovered by the inventors to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### EXAMPLE 1

An aerosol formulation may be prepared with the following composition:

| Sr. No. | Ingredient | % by weight |
|---|---|---|
| 1 | Beclometasone dipropionate | 0.172 |
| 2 | Formoterol fumarate dihydrate | 0.010 |
| 3 | Ethanol | 9.5 |
| 4 | Maleic acid | 0.003 |
| 5 | HFA134a | q.s |

This solution formulation is filled under pressure into a canister fitted with a metering valve having a 50 μl metering chamber.

### Manufacturing Process 1

1. Weighed quantity of beclometasone dipropionate along with formoterol fumarate dihydrate are dissolved in between 30-70% ethanol. Stir till a clear solution is obtained.
2. Weighed quantity of maleic acid is dissolved in the remaining ethanol. Stir the solution.
3. The solution of step 1 is mixed with solution of step 2 and charge the mixture into the batch vessel.
4. Charge part of the HFA134a propellant into the batching vessel and stir to homogenize.
5. Fill concentrate into canisters.
6. Fill balance of propellant

### EXAMPLE 2

An aerosol formulation may be prepared with the following composition:

| Sr. No. | Ingredient | % by weight |
|---|---|---|
| 1 | Beclometasone dipropionate | 0.271 |
| 2 | Formoterol fumarate dihydrate | 0.008 |
| 3 | Ethanol | 9.5 |
| 4 | Maleic acid | 0.002 |
| 5 | HFA134a | q.s |

This solution formulation is filled under pressure into a canister fitted with a metering valve having a 63 μl metering chamber. The formulation is manufactured using Manufacturing Process 1 described above.

### EXAMPLE 3

An aerosol formulation may be prepared with the following composition:

| Sr. No. | Ingredient | % by weight |
|---|---|---|
| 1 | Beclometasone dipropionate | 0.172 |
| 2 | Formoterol fumarate dihydrate | 0.010 |
| 3 | Ethanol | 12 |
| 4 | Maleic acid | 0.003 |
| 5 | Water | 0.5 |
| 6 | HFA134a | q.s |

This solution formulation is filled under pressure into a canister fitted with a metering valve having a 50 μl metering chamber.

### Manufacturing Process 2

1. Weighed quantity of beclometasone dipropionate along with formoterol fumarate dihydrate are dissolved in between 30-70% ethanol. Stir till a clear solution is obtained.
2. Weighed quantity of maleic acid is dissolved in water and the remaining ethanol. Stir the solution.
3. The solution of step 1 is mixed with solution of step 2 and charge the mixture into the batch vessel.
4. Charge part of the HFA134a propellant into the batching vessel and stir to homogenize.
5. Fill concentrate into canisters.
6. Fill rest of propellant

### EXAMPLE 4

An aerosol formulation may be prepared with the following composition:

| Sr. No. | Ingredient | % by weight |
|---|---|---|
| 1 | Beclometasone dipropionate | 0.271 |
| 2 | Formoterol fumarate dihydrate | 0.010 |
| 3 | Ethanol | 12 |
| 4 | Maleic acid | 0.002 |
| 5 | Water | 0.5 |
| 6 | HFA134a | q.s |

This solution formulation is filled into a canister fitted with a metering valve having a 63 μl metering chamber followed by the addition of the propellant.

### Manufacturing Process 3

1. Weighed quantity of beclometasone dipropionate along with formoterol fumarate dihydrate are dissolved in ethanol. Stir till a clear solution is obtained.
2. Weighed quantity of maleic acid dissolved in water and the remaining ethanol. Stir the solution.
3. The solution of step 1 are mixed with solution of step 2 and stirred to homogenize.
4. Fill concentrate into canisters.
5. Fill the required propellant into the canisters

### EXAMPLE 5

An aerosol formulation may be prepared with the following composition:

| Sr. No. | Ingredient | % by weight |
|---|---|---|
| 1 | Beclometasone dipropionate | 0.172 |
| 2 | Formoterol fumarate dihydrate | 0.010 |
| 3 | Ethanol | 16 |
| 4 | HFA134a | q.s |

This solution formulation is filled under pressure into a canister fitted with a metering valve having a 50 μl metering chamber.

### Manufacturing Process 4

1. Weighed quantity of beclometasone dipropionate along with formoterol fumarate dihydrate are dissolved in ethanol. Stir till a clear solution is obtained.
2. Charge part of the HFA134a propellant into the batching vessel and stir to homogenize.
3. Fill concentrate into canisters.
4. Fill balance of propellant.

### EXAMPLE 6

A design of experiments was performed with formulations prepared with the following compositions:

| Sr. No. | Ingredient | % by weight |
|---|---|---|
| 1 | Beclometasone dipropionate | 0.172 |
| 2 | Formoterol fumarate dihydrate | 0.010 |
| 3 | Ethanol | 12 |
| 4 | Maleic acid | 0.0024-0.0033 |
| 5 | Water | 0-0.75 |
| 6 | HFA134a | q.s |

Each solution formulation is filled into a canister fitted with a metering valve having a 50 μl metering chamber followed by the addition of the propellant. The formulations are manufactured using Manufacturing Process 3 described above.

### Brief Procedure for Determination of Assay/Total Degradation Product

Assay and related substances determinations are carried out using a Liquid Chromatograph (LC) by recovering the drug substances from the canisters with appropriate diluent systems composed of water and organic solvents. The test samples are run on the LC using a C18 column with a suitable organic: aqueous mobile phase. Peak identification and quantitation is carried out from the resulting chromatography.

### Assay Data (Formoterol Fumarate)

TABLE 1
Low strength

| Example 1 | | | | Example 3 | | | | | |
|---|---|---|---|---|---|---|---|---|---|
| | ACC | CRT | 5° C. | | ACC | CRT | | 5° C. | |
| Initial | 1M | 1M | 2M | Initial | 1M | 1M | 3M | 2M | 3M |
| 100.0% | 92.8% | 99.4% | 98.5% | 100.0% | 96.6% | 98.8% | 99.6% | 97.7% | 99.3% |

TABLE 2
High strength

| Example 2 | | | | | Example 4 | | |
|---|---|---|---|---|---|---|---|
| | ACC | CRT | | 5° C. | | ACC | 5° C. |
| Initial | 1M | 1M | 3M | 3M | Initial | 1M | 1M |
| 100.0% | 89.4% | 98.2% | 99.4% | 102.2% | 100.0% | 93.3% | 99.6% |

As evident from Table 1 and 2, the Assay data demonstrates the stability of the pharmaceutical composition of formoterol in the presence of organic acid. In addition, the data further demonstrate the surprising added benefit of water to form a stable pharmaceutical composition of formoterol.

Total Impurity Data (Formoterol Fumarate)

The examples presented above were subjected to various storage conditions to evaluate the chemical stability of these compositions. The accelerated storage condition (ACC) is the harshest, with the samples being subjected to 40° C. and 75% Relative Humidity. Data after 1 month storage at the ACC condition is a good indicator of stability of the composition.

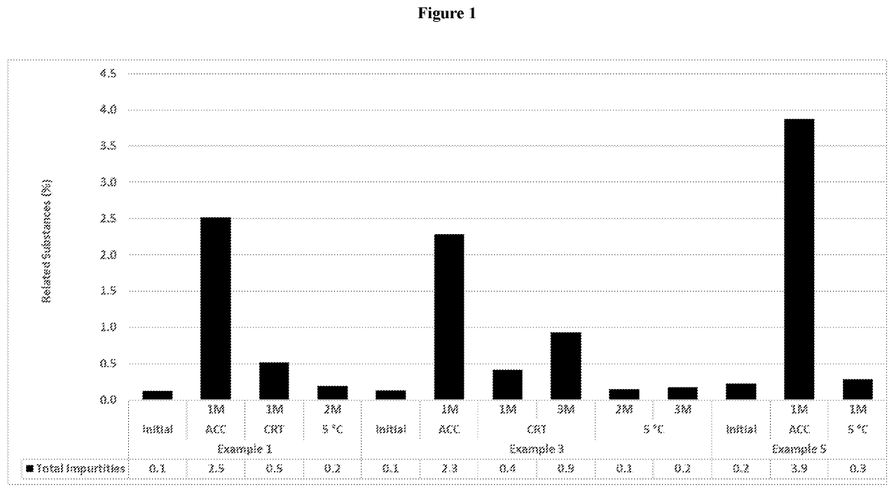

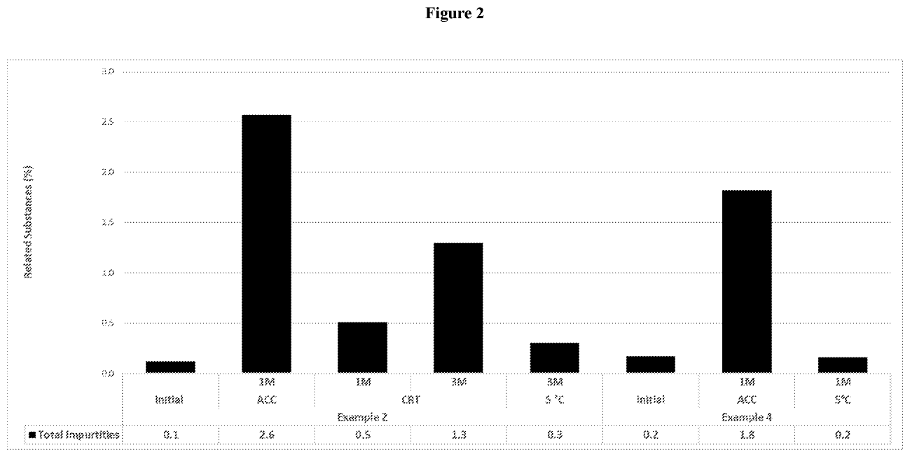

As evident from FIGS. 1 and 2, the ACC data demonstrates that the composition having a solution of formoterol in the presence of an organic acid, specifically maleic acid was found to be stable in comparison to a composition having a solution of formoterol without organic acid viz., due to significant reduction in the total degradation of the composition with organic acid. Further, the composition, additionally having water, surprisingly demonstrates added benefit in reducing the total degradation of formoterol.

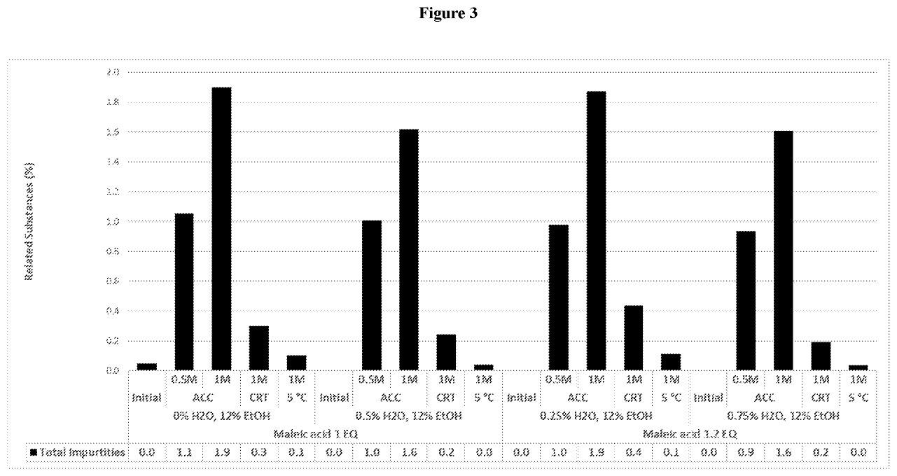

As evident from FIG. 3, the ACC data demonstrates that the composition having a solution of formoterol in the presence of an organic acid, specifically maleic acid was found to be stable due to significant reduction in the total degradation of the composition with organic acid.

Further, the composition, additionally having water in varying percentage i.e., 0.5%, 0.75% surprisingly demonstrates added benefit in reducing the total degradation of formoterol.

### Abbreviation

ACC is ICH Accelerated storage condition (40 C/75% RH)
CRT is ICH long term storage (25 C/60% RH)

1. A pharmaceutical composition comprising β2-agonist, a propellant, a co-solvent and organic acid(s).

2. The pharmaceutical composition of claim 1, further comprises water.

3. The pharmaceutical composition of claim 2, wherein the water is present in the amount of up to 3% by weight.

4. The pharmaceutical composition of claim 1, wherein the β2-agonist drug has benzylic hydroxyl group having susceptibility to substitution by nucleophilic species in the formulation via SN1 or SN2 reactions.

5. The pharmaceutical composition of claim 4, wherein the β2-agonist drug is selected from a group consisting of formoterol, vilanterol, indacaterol and salmeterol or pharmaceutically acceptable salts thereof

6. The pharmaceutical composition of claim 1, wherein the β2 agonist is present in the composition in an amount from about 0.001% to about 0.2% by weight.

7. The pharmaceutical composition of claim 1, wherein the propellant is selected from HFA 134a, HFA 227ea and HFA 152a or mixtures thereof.

8. The pharmaceutical composition of claim 1, wherein the co-solvent is selected from a group consisting of dichloromethane, chloroform, ethylacetate, N-methyl pyrrolidone, benzylalcohol, isopropylacetate, acetonitrile, tetrahydrofuran, isopropanol, methanol and ethanol or mixtures thereof.

9. The pharmaceutical composition of claim 1, wherein the co-solvent is present in the composition in an amount from about 1% to about 40% by weight.

10. The pharmaceutical composition of claim 1, wherein the co-solvent is ethanol.

11. The pharmaceutical composition of claim 10, wherein the co-solvent ethanol is present in concentration of about 4 to about 20% by weight.

12. The pharmaceutical composition of claim 1, wherein the organic acid is selected from a group consisting of maleic acid, fumaric acid, citric acid, acetic acid, xinafoic acid, oxalic acid, lactic acid, 2-methyl propionic acid, malic acid, butanoic acid, tartaric acid, propionic acid, pentanoic acid, succinic acid, glycolic acid, hexanoic acid, malonic acid, glutaric acid, formic acid, adipic acid, ascorbic acid, benzoic acid and glucuronic acid or mixtures thereof.

13. The pharmaceutical composition of claim 1, further comprises second active agent which is a corticosteroid or long acting muscarinic receptor antagonists.

14. The pharmaceutical composition of claim 13, wherein the corticosteroid is selected from a group consisting of beclometasone diporpionate, budesonide, ciclesonide, flunisolide, fluticasone propionate, fluticasone furoate, mometasone, triamcinolone acetate and prednisolone.

15. The pharmaceutical composition of claim 13, wherein the corticosteroid is present in the composition in an amount from about 0.001% to about 0.6% by weight.

16. The pharmaceutical composition of claim 13, wherein the long acting muscarinic receptor antagonist is selected from a group consisting of umeclidinium, aclidinium, glycopyrronium, ipratropium, oxitropium and tiotropium or pharmaceutically acceptable salts thereof.

17. The pharmaceutical composition of claim 1, is filled in a container having part or all of its internal metallic surfaces made of stainless steel, anodised aluminium or lined with an inert organic coating.

18. A method of use of a pharmaceutical composition according to claim 1 for the treatment or prophylaxis of a respiratory disorder.

19. A method of use of a pharmaceutical composition according to claim 1 for the treatment or prophylaxis of asthma, COPD, rhinitis or as adjunct therapy for cystic fibrosis, non-cystic fibrosis bronchiectasis, lung infections or pulmonary fibrosis.
